(19) United States
(12) Patent Application Publication
(10) Pub. No.: US 2022/0389452 A1
(43) Pub. Date: Dec. 8, 2022

(54) GENE THERAPY COMPOSITION AND TREATMENT OF RIGHT VENTRICULAR ARRYTHMOGENIC CARDIOMYOPATHY

(71) Applicant: UCL BUSINESS LTD, London (GB)

(72) Inventors: Thomas Voit, London (GB); Julie Dumonceaux, London (GB); Perry Elliott, London (GB); Virginie Mariot, London (GB)

(21) Appl. No.: 17/761,677

(22) PCT Filed: Sep. 21, 2020
(86) PCT No.: PCT/EP2020/076290

§ 371 Date: Mar. 18, 2022

Related U.S. Application Data
(60) Provisional application No. 62/903,103, filed on Sep. 20, 2019.

(51) Int. Cl. C12N 15/86

(52) U.S. Cl. CPC C12N 15/86; A61K 48/00

(57) ABSTRACT

Disclosed are a composition and method of treating or preventing cardiomyopathy in a human subject. In some embodiments, the method comprises delivering a therapeutic dose of a gene therapy vector to cardiomyocytes of the human subject, wherein the gene therapy vector comprises a nucleic acid sequence encoding for PKP2.

### CROSS-REFERENCE TO RELATED APPLICATION(S)

This application claims the benefit of priority of U.S. Provisional Patent Application Ser. No. 62/903,103, filed on Sep. 20, 2019, the disclosure of which is hereby incorporated by reference herein in its entirety.

### FIELD OF THE INVENTION

The present invention relates to the treatment of cardiac diseases (e.g., cardiac myopathies), and, more specifically, to gene therapy methods and pharmaceutical compositions for the treatment of cardiomyopathy.

### BACKGROUND OF THE INVENTION

Despite pharmacologic advances in the treatment of various heart conditions, such as heart failure, mortality, and morbidity remain unacceptably high. Furthermore, certain therapeutic approaches are not suitable for many patients (e.g., ones who have an advanced heart failure condition associated with other co-morbid diseases). Alternative approaches, such as gene therapy and cell therapy, have attracted increased attention due to their potential to be uniquely tailored and efficacious in addressing the root cause pathogenesis of many cardiac diseases.

### OBJECTS AND SUMMARY OF THE INVENTION

It is an object of the present invention to provide methods of delivering therapeutic polynucleotide sequences to cardiomyocytes of a human subject.

It is a further object of certain embodiments of the present invention to vectorize a polynucleotide sequence encoding for plakophilin-2 (PKP2) protein in a viral vector, such as an adeno-associated virus.

It is a further object of certain embodiments of the present invention to utilize gene therapy methods for correcting haploinsufficiency in PKP2-mutated cardiomyocytes.

The above objects and others are met by the present invention which in certain embodiments is directed to a method of treating or preventing cardiomyopathy in a human subject. The method includes, e.g., delivering a therapeutic dose of a gene therapy vector to cardiomyocytes of the human subject, wherein the gene therapy vector comprises a nucleic acid sequence encoding for PKP2.

In some embodiments, the gene therapy vector comprises a viral vector. In some embodiments, the viral vector comprises one or more of AAV1, AAV2, AAV3, AAV4, AAV5, AAV6, AAV7, AAV8, AAV9, AAV10, AAV11, AAV12, variations thereof, and combinations thereof. In some embodiments, the viral vector comprises AAV6 or AAV9.

In some embodiments, the therapeutic dose is effective to treat or prevent arrhythmogenic right ventricular cardiomyopathy (ARVC) by effecting the production of PKP2 protein by the cardiomyocytes of the human subject.

Certain other embodiments are directed to a gene therapy vector adapted for expressing a nucleic acid sequence within cardiomyocytes of a human subject. The nucleic acid sequence comprises, e.g., a first sequence encoding for a PKP2 protein and a second sequence comprising a promoter. In some embodiments, the gene therapy vector comprises a viral vector. In some embodiments, the viral vector comprises one or more of AAV1, AAV2, AAV3, AAV4, AAV5, AAV6, AAV7, AAV8, AAV9, AAV10, AAV11, AAV12, variations thereof, and combinations thereof. In some embodiments, the viral vector comprises AAV6 or AAV9.

In some embodiments, the promoter comprises TNNT2.

### Definitions

As used herein, the singular forms “a,” “an,” and “the” include plural references unless the context clearly indicates otherwise. Thus, for example, reference to “a drug” includes a single drug as well as a mixture of two or more different drugs; and reference to a “viral vector” includes a single viral vector as well as a mixture of two or more different viral vectors, and the like.

Also as used herein, “about,” when used in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

Also as used herein, “polynucleotide” has its ordinary and customary meaning in the art and includes any polymeric nucleic acid such as DNA or RNA molecules, as well as chemical derivatives known to those skilled in the art. Polynucleotides include not only those encoding a therapeutic protein, but also include sequences that can be used to decrease the expression of a targeted nucleic acid sequence using techniques known in the art (e.g., antisense, interfering, or small interfering nucleic acids). Polynucleotides can also be used to initiate or increase the expression of a targeted nucleic acid sequence or the production of a targeted protein within cells of the cardiovascular system. Targeted nucleic acids and proteins include, but are not limited to, nucleic acids and proteins normally found in the targeted tissue, derivatives of such naturally occurring nucleic acids or proteins, naturally occurring nucleic acids or proteins not normally found in the targeted tissue, or synthetic nucleic acids or proteins. One or more polynucleotides can be used in combination, administered simultaneously and/or sequentially, to increase and/or decrease one or more targeted nucleic acid sequences or proteins.

Also as used herein, “exogenous” nucleic acids or genes are those that do not occur in nature in the vector utilized for nucleic acid transfer; e.g., not naturally found in the viral vector, but the term is not intended to exclude nucleic acids encoding a protein or polypeptide that occurs naturally in the patient or host.

Also as used herein, “cardiac cell” includes any cell of the heart that is involved in maintaining a structure or providing a function of the heart such as a cardiac muscle cell, a cell of the cardiac vasculature, or a cell present in a cardiac valve. Cardiac cells include cardio myocytes (having both normal and abnormal electrical properties), epithelial cells, endothelial cells, fibroblasts, cells of the conducting tissue, cardiac pace making cells, and neurons.

Also as used herein, “adeno-associated virus” or “AAV” encompasses all subtypes, serotypes, and pseudotypes, as well as naturally occurring and recombinant forms. A variety of AAV serotypes and strains are known in the art and are publicly available from sources, such as the ATCC and academic or commercial sources. Alternatively, sequences from AAV serotypes and strains which are published and/or available from a variety of databases may be synthesized using known techniques.

Also as used herein, “serotype” refers to an AAV which is identified by and distinguished from other AAVs based on capsid protein reactivity with defined antisera. There are at least twelve known serotypes of human AAV, including AAV1 through AAV12, however additional serotypes continue to be discovered, and use of newly discovered serotypes are contemplated.

Also as used herein, “pseudotyped” AAV refers to an AAV that contains capsid proteins from one serotype and a viral genome including 5′ and 3′ inverted terminal repeats (ITRs) of a different or heterologous serotype. A pseudotyped recombinant AAV (rAAV) would be expected to have cell surface binding properties of the capsid serotype and genetic properties consistent with the ITR serotype. A pseudotyped rAAV may comprise AAV capsid proteins, including VP1, VP2, and VP3 capsid proteins, and ITRs from any serotype AAV, including any primate AAV serotype from AAV1 through AAV12, as long as the capsid protein is of a serotype heterologous to the serotype(s) of the ITRs. In a pseudotyped rAAV, the 5′ and 3′ ITRs may be identical or heterologous. Pseudotyped rAAV are produced using standard techniques described in the art.

Also as used herein, a “chimeric” rAAV vector encompasses an AAV vector comprising heterologous capsid proteins; that is, a rAAV vector may be chimeric with respect to its capsid proteins VP1, VP2, and VP3, such that VP1, VP2, and VP3 are not all of the same serotype AAV. A chimeric AAV as used herein encompasses AAV wherein the capsid proteins VP1, VP2, and VP3 differ in serotypes, including for example but not limited to capsid proteins from AAV1 and AAV2; are mixtures of other parvo virus capsid proteins or comprise other virus proteins or other proteins, such as for example, proteins that target delivery of the AAV to desired cells or tissues. A chimeric rAAV as used herein also encompasses an rAAV comprising chimeric 5′ and 3′ ITRs.

Also as used herein, a “pharmaceutically acceptable excipient or carrier” refers to any inert ingredient in a composition that is combined with an active agent in a formulation. A pharmaceutically acceptable excipient can include, but is not limited to, carbohydrates (such as glucose, sucrose, or dextrans), antioxidants (such as ascorbic acid or glutathione), chelating agents, low-molecular weight proteins, high-molecular weight polymers, gel-forming agents, or other stabilizers and additives. Other examples of a pharmaceutically acceptable carrier include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

Also as used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

Also as used herein, a “subject” encompasses the definition of the term “patient” and does not exclude individuals who are otherwise healthy.

Also as used herein, “treatment of” and “treating” include the administration of a drug with the intent to lessen the severity of or prevent a condition, e.g., heart disease.

Also as used herein, “prevention of” and “preventing” include the avoidance of the onset of a condition, e.g., heart disease.

Also as used herein, a “condition” or “conditions” refers to those medical conditions, such as heart disease, that can be treated, mitigated, or prevented by administration to a subject of an effective amount of a drug.

Also as used herein, an “effective amount” refers to the amount of a drug that is sufficient to produce a beneficial or desired effect at a level that is readily detectable by a method commonly used for detection of such an effect. In some embodiments, such an effect results in a change of at least 10% from the value of a basal level where the drug is not administered. In other embodiments, the change is at least 20%, 50%, 80%, or an even higher percentage from the basal level. As will be described below, the effective amount of a drug may vary from subject to subject, depending on age, general condition of the subject, the severity of the condition being treated, the particular drug administered, and the like. An appropriate “effective” amount in any individual case may be determined by one of ordinary skill in the art by reference to the pertinent texts and literature and/or by using routine experimentation.

Also as used herein, an “active agent” refer to any material that is intended to produce a therapeutic, prophylactic, or other intended effect, whether or not approved by a government agency for that purpose.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

Arrhythmogenic right ventricular cardiomyopathy (ARVC) is a primary heart muscle disorder and a major cause of sudden cardiac death (SCD) in young individuals. It is characterized by myocardial degeneration and fibro-fatty replacement of the myocardium, which can be present in the right and/or left ventricle and ultimately lead to progressive heart failure. The clinical cardiac phenotype is characterized by the presence of typical electrocardiographic abnormalities, an increased burden of ventricular arrhythmias, and extensive myocardial scarring on cardiac magnetic resonance imaging.

ARVC is familial in approximately 50% of cases and is usually inherited as an autosomal dominant trait. About 30% of patients of Caucasian descent carry dominant mutations in the PKP2 gene. The majority of mutations result in aberrant or truncated protein resulting from insertion-deletion, nonsense, or splice site mutations, resulting in haploinsufficiency.

ARVC is considered a disease of the desmosome, the electron-dense structure providing mechanical attachment between cardiomyocytes. PKP2 is one among several genes which form part of the desmosomal protein complex and where mutations leading to ARVC have been identified. Lack of PKP2 protein through haploinsufficiency destabilizes the desmosomal protein complex with mechanical and signaling consequences.

The mechanical component is highlighted in vitro by the abnormal gene expression pattern caused by lack of PKP2 protein under mechanical stress conditions involving down-regulation of several extracellular matrix genes such as different collagens and strong up-regulation of fibril-forming collagens, fibronectin, and other pro-fibrotic markers such as TIMP1. In pre-clinical and clinical contexts, this is mirrored by exacerbation of ARVC by exercise in PKP2-mouse models and by the detrimental effects of exercise on the phenotype in humans, such as in athletes. At the signaling level, lack of plakophilin causes translocation of plakoglobin to the nucleus, which leads to reduction of canonical Wnt/b-catenin signaling and increased expression of fibrogenic and adipogenic genes.

The two main forms of PKP2 include PKP2 isoform 2a (SEQ ID NO: 2) and PKP2 isoform 2b (SEQ ID NO: 4). The protein coding portion of the PKP2 gene for PKP2 isoform 2a is contained in a 2764bp cDNA sequence (GenBank: BC126199.1; SEQ ID NO:1), which can be vectorized in an AAV by virtue of the present invention. As used herein, “PKP2” or “PKP2 protein,” unless otherwise stated or implied from the context, should be interpreted to encompass the isoforms of PKP2, including PKP2 isoform 2a and PKP2 isoform 2b.

Certain embodiments may correct haploinsufficiency of the PKP2 protein by substituting a normal allele via AAV9-TNNT2-PKP2-mediated gene transfer. In certain embodiments, the compositions and methods of the present invention may be capable of, e.g., (1) localizing the PKP2 protein correctly to the desmosome; and (2) correcting the haploinsufficiency in PKP2-mutated human induced pluripotent stem cell-derived cardiomyocytes (iPSC-CMs) and consequently correcting the desmosomal protein complex. Certain embodiments are also contemplated to result in complete or near-complete PKP2 deficiency in iPSC-CMs carrying two pathogenic mutations in trans. A non-limiting illustrative embodiment for testing the delivery of PKP2 polynucleotides to cardiomyocytes include: (1) vectorizing PKP2 using a TNNT2 promoter into AAV9 and/or AAV6; creating create iPSC-CMs carrying PKP2 mutations (either 1 mutation or 2 mutations in trans); transducing 2D PKP2-mutated cardiomyocyte cultures (carrying 1 or 2 mutations) with AAV6-PKP2 or AAV9-PKP2 in vitro and testing subcellular localization into desmosomes; testing molecular and physiological data including cell size, contractility, and transcriptome analysis.

Although numerous embodiments herein are described with respect to PKP2 protein, it is to be understood that the expression of additional proteins (e.g., sarcomeric proteins) is contemplated. Exemplary proteins in addition to PKP2 may include, without limitations, one or more of SERCA2, MYBPC3, MYH7, MYL3, MYL2, ACTC1, TPM1, TNNT2, TNNI3, TTN, FHL1, ALPK3, dystrophin, FKRP, variants thereof, or combinations thereof. The protein or proteins used may also be functional variants of the proteins mentioned herein and may exhibit a significant amino acid sequence identity compared to the original protein. For instance, the amino acid identity may amount to at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In this context, the term “functional variant” means that the variant of the protein is capable of, partially or completely, fulfilling the function of the naturally occurring corresponding protein. Functional variants of a protein may include, for example, proteins that differ from their naturally occurring counterparts by one or more amino acid substitutions, deletions or additions.

The amino acid substitutions can be conservative or non-conservative. It is preferred that the substitutions are conservative substitutions, i.e., a substitution of an amino acid residue by an amino acid of similar polarity, which acts as a functional equivalent. Preferably, the amino acid residue used as a substitute is selected from the same group of amino acids as the amino acid residue to be substituted. For example, a hydrophobic residue can be substituted with another hydrophobic residue, or a polar residue can be substituted with another polar residue having the same charge. Functionally homologous amino acids, which may be used for a conservative substitution comprise, for example, non-polar amino acids such as glycine, valine, alanine, isoleucine, leucine, methionine, proline, phenylalanine, and tryptophan. Examples of uncharged polar amino acids comprise serine, threonine, glutamine, asparagine, tyrosine and cysteine. Examples of charged polar (basic) amino acids comprise histidine, arginine, and lysine. Examples of charged polar (acidic) amino acids comprise aspartic acid and glutamic acid.

Also considered as variants are proteins that differ from their naturally occurring counterparts by one or more (e.g., 2, 3, 4, 5, 10, or 15) additional amino acids. These additional amino acids may be present within the amino acid sequence of the original protein (i.e., as an insertion), or they may be added to one or both termini of the protein. Basically, insertions can take place at any position if the addition of amino acids does not impair the capability of the polypeptide to fulfill the function of the naturally occurring protein in the treated subject. Moreover, variants of proteins also comprise proteins in which, compared to the original polypeptide, one or more amino acids are lacking. Such deletions may affect any amino acid position provided that it does not impair the ability to fulfill the normal function of the protein.

Finally, variants of cardiac sarcomeric proteins (e.g., PKP2) also refer to proteins that differ from the naturally occurring protein by structural modifications, such as modified amino acids. Modified amino acids are amino acids which have been modified either by natural processes, such as processing or post-translational modifications, or by chemical modification processes known in the art. Typical amino acid modifications comprise phosphorylation, glycosylation, acetylation, O-linked N-acetylglucosamination, glutathionylation, acylation, branching, ADP ribosylation, crosslinking, disulfide bridge formation, formylation, hydroxylation, carboxylation, methylation, demethylation, amidation, cyclization, and/or covalent or non-covalent bonding to phosphotidylinositol, flavine derivatives, lipoteichonic acids, fatty acids, or lipids.

The therapeutic polynucleotide sequence encoding the target protein may be administered to the subject to be treated in the form of a gene therapy vector, i.e., a nucleic acid construct which comprises the coding sequence, including the translation and termination codons, next to other sequences required for providing expression of the exogenous nucleic acid such as promoters, kozak sequences, polyA signals, and the like.

For example, the gene therapy vector may be part of a mammalian expression system. Useful mammalian expression systems and expression constructs are commercially available. Also, several mammalian expression systems are distributed by different manufacturers and can be employed in the present invention, such as plasmid- or viral vector based systems, e.g., LENTI-Smart™ (InvivoGen), GenScript™ Expression vectors, pAdVAntage™ (Promega), ViraPower™ Lentiviral, Adenoviral Expression Systems (Invitrogen), and adeno-associated viral expression systems (Cell Biolabs).

Gene therapy vectors for expressing an exogenous therapeutic polynucleotide sequence of the invention can be, for example, a viral or non-viral expression vector, which is suitable for introducing the exogenous therapeutic polynucleotide sequence into a cell for subsequent expression of the protein encoded by said nucleic acid. The expression vector can be an episomal vector, i.e., one that is capable of self-replicating autonomously within the host cell, or an integrating vector, i.e., one which stably incorporates into the genome of the cell. The expression in the host cell can be constitutive or regulated (e.g., inducible).

In a certain embodiment, the gene therapy vector is a viral expression vector. Viral vectors for use in the present invention may comprise a viral genome in which a portion of the native sequence has been deleted in order to introduce a heterogeneous polynucleotide without destroying the infectivity of the virus. Due to the specific interaction between virus components and host cell receptors, viral vectors are highly suitable for efficient transfer of genes into target cells. Suitable viral vectors for facilitating gene transfer into a mammalian cell can be derived from different types of viruses, for example, from an AAV, an adenovirus, a retrovirus, a herpes simplex virus, a bovine papilloma virus, a lentivirus, a vaccinia virus, a polyoma virus, a sendai virus, orthomyxovirus, paramyxovirus, papovavirus, picornavirus, pox virus, alphavirus, or any other viral shuttle suitable for gene therapy, variations thereof, and combinations thereof.

“Adenovirus expression vector” or “adenovirus” is meant to include those constructs containing adenovirus sequences sufficient (a) to support packaging of the therapeutic polynucleotide sequence construct, and/or (b) to ultimately express a tissue and/or cell-specific construct that has been cloned therein. In one embodiment of the invention, the expression vector comprises a genetically engineered form of adenovirus. Knowledge of the genetic organization of adenovirus, a 36 kilobase (kb), linear, double-stranded DNA virus, allows substitution of large pieces of adenoviral DNA with foreign sequences up to 7 kb.

Adenovirus growth and manipulation is known to those of skill in the art, and exhibits broad host range in vitro and in vivo. This group of viruses can be obtained in high titers, e.g., 10⁹ to 10¹¹ plaque-forming units per mL, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and/or therapeutic potential as in vivo gene transfer vectors.

Retroviruses (also referred to as “retroviral vector”) may be chosen as gene delivery vectors due to their ability to integrate their genes into the host genome, transferring a large amount of foreign genetic material, infecting a broad spectrum of species and cell types and for being packaged in special cell-lines.

The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

The retrovirus can be derived from any of the subfamilies. For example, vectors from Murine Sarcoma Virus, Bovine Leukemia, Virus Rous Sarcoma Virus, Murine Leukemia Virus, Mink-Cell Focus-Inducing Virus, Reticuloendotheliosis Virus, or Avian Leukosis Virus can be used. The skilled person will be able to combine portions derived from different retroviruses, such as LTRs, tRNA binding sites, and packaging signals to provide a recombinant retrovirus. These retroviruses are then normally used for producing transduction competent retroviral vector particles. For this purpose, the vectors are introduced into suitable packaging cell lines. Retroviruses can also be constructed for site-specific integration into the DNA of the host cell by incorporating a chimeric integrase enzyme into the retroviral particle.

Because herpes simplex virus (HSV) is neurotropic, it has generated considerable interest in treating nervous system disorders. Moreover, the ability of HSV to establish latent infections in non-dividing neuronal cells without integrating into the host cell chromosome or otherwise altering the host cell's metabolism, along with the existence of a promoter that is active during latency makes HSV an attractive vector. And though much attention has focused on the neurotropic applications of HSV, this vector also can be exploited for other tissues given its wide host range.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient multiplicity of infection (MOI) and in a lessened need for repeat dosing. Avirulent variants of HSV have been developed and are readily available for use in gene therapy contexts.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu, and nef are deleted making the vector biologically safe.

Lentiviral vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome. Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus results in a level of expression that is higher than that of conventional vectors, with the transgenes accumulating at 10% or more of the infected cell's protein in 24 hours.

The empty capsids of papovaviruses, such as the mouse polyoma virus, have received attention as possible vectors for gene transfer. The use of empty polyoma was first described when polyoma DNA and purified empty capsids were incubated in a cell-free system. The DNA of the new particle was protected from the action of pancreatic DNase. The reconstituted particles were used for transferring a transforming polyoma DNA fragment to rat FIII cells. The empty capsids and reconstituted particles consist of all three of the polyoma capsid antigens VP1, VP2 and VP3.

AAVs are parvoviruses belonging to the genus Dependovirus. They are small, nonenveloped, single-stranded DNA viruses which require a helper virus in order to replicate. Co-infection with a helper virus (e.g., adenovirus, herpes virus, or vaccinia virus) is necessary in order to form functionally complete AAV virions. In vitro, in the absence of co-infection with a helper virus, AAV establishes a latent state in which the viral genome exists in an episomal form, but infectious virions are not produced. Subsequent infection by a helper virus “rescues” the genome, allowing it to be replicated and packaged into viral capsids, thereby reconstituting the infectious virion. Recent data indicate that in vivo both wild type AAV and recombinant AAV predominantly exist as large episomal concatemers. In one embodiment, the gene therapy vector used herein is an AAV vector. The AAV vector may be purified, replication incompetent, pseudotyped rAAV particles.

AAV are not associated with any known human diseases, are generally not considered pathogenic, and do not appear to alter the physiological properties of the host cell upon integration. AAV can infect a wide range of host cells, including non-dividing cells, and can infect cells from different species. In contrast to some vectors, which are quickly cleared or inactivated by both cellular and humoral responses, AAV vectors have been shown to induce persistent transgene expression in various tissues in vivo. The persistence of recombinant AAV-mediated transgenes in non-diving cells in vivo may be attributed to the lack of native AAV viral genes and the vector's ITR-linked ability to form episomal concatemers.

AAV is an attractive vector system for use in the cell transduction of the present invention as it has a high frequency of persistence as an episomal concatemer and it can infect non-dividing cells, including cardiomyocytes, thus making it useful for delivery of genes into mammalian cells, for example, in tissue culture and in vivo.

Typically, rAAV is made by cotransfecting a plasmid containing the gene of interest flanked by the two AAV terminal repeats and/or an expression plasmid containing the wild-type AAV coding sequences without the terminal repeats, for example pIM45. The cells are also infected and/or transfected with adenovirus and/or plasmids carrying the adenovirus genes required for AAV helper function. Stocks of rAAV made in such fashion are contaminated with adenovirus, which must be physically separated from the rAAV particles (for example, by cesium chloride density centrifugation or column chromatography). Alternatively, adenovirus vectors containing the AAV coding regions and/or cell lines containing the AAV coding regions and/or some or all of the adenovirus helper genes could be used. Cell lines carrying the rAAV DNA as an integrated provirus can also be used.

Multiple serotypes of AAV exist in nature, with at least twelve serotypes (AAV1-AAV12). Despite the high degree of homology, the different serotypes have tropisms for different tissues. Upon transfection, AAV elicits only a minor immune reaction (if any) in the host. Therefore, AAV is highly suited for gene therapy approaches.

The present disclosure may be directed in some embodiments to a drug comprising an AAV vector that is one or more of AAV1, AAV2, AAV3, AAV4, AAV5, AAV6, AAV7, AAV8, AAV9, AAV10, AAV11, AAV12, ANC AAV, chimeric AAV derived thereof, variations thereof, and combinations thereof, which will be even better suitable for high efficiency transduction in the tissue of interest. In certain embodiments, the gene therapy vector is an AAV serotype 1 vector. In certain embodiments, the gene therapy vector is an AAV serotype 2 vector. In certain embodiments, the gene therapy vector is an AAV serotype 3 vector. In certain embodiments, the gene therapy vector is an AAV serotype 4 vector. In certain embodiments, the gene therapy vector is an AAV serotype 5 vector. In certain embodiments, the gene therapy vector is an AAV serotype 6 vector. In certain embodiments, the gene therapy vector is an AAV serotype 7 vector. In certain embodiments, the gene therapy vector is an AAV serotype 8 vector. In certain embodiments, the gene therapy vector is an AAV serotype 9 vector. In certain embodiments, the gene therapy vector is an AAV serotype 10 vector. In certain embodiments, the gene therapy vector is an AAV serotype 11 vector. In certain embodiments, the gene therapy vector is an AAV serotype 12 vector.

A suitable dose of AAV for humans may be in the range of about 1×10⁸ vg/kg to about 3×10¹⁴ vg/kg, about 1×10⁸ vg/kg, about 1×10⁹ vg/kg, about 1×10¹° vg/kg, about 1×10¹¹ vg/kg, about 1×10¹² vg/kg, about 1×10¹³ vg/kg, or about 1×10¹⁴ vg/kg. The total amount of viral particles or DRP is, is about, is at least, is at least about, is not more than, or is not more than about, 5×10¹⁵ vg/kg, 4×10¹⁵ vg/kg, 3×10¹⁵ vg/kg, 2×10¹⁵ vg/kg, 1×10¹⁵ vg/kg, 9×10¹⁴ vg/kg, 8×10¹⁴ vg/kg, 7×10¹⁴ vg/kg, 6×10¹⁴ vg/kg, 5×10¹⁴ vg/kg, 4×10¹⁴ vg/kg, 3×10¹⁴ vg/kg, 2×10¹⁴ vg/kg, 1×10¹⁴ vg/kg, 9×10¹³ vg/kg, 8×10¹³ vg/kg, 7×10¹³ vg/kg, 6×10¹³ vg/kg, 5×10¹³ vg/kg, 4×10¹³ vg/kg, 3×10¹³ vg/kg, 2×10¹³ vg/kg, 1×10¹³ vg/kg, 9×10¹² vg/kg, 8×10¹² vg/kg, 7×10¹² vg/kg, 6×10¹² vg/kg, 5×10¹² vg/kg, 4×10¹² vg/kg, 3×10¹² vg/kg, 2×10¹² vg/kg, 1×10¹² vg/kg, 9×10¹¹ vg/kg, 8×10″ vg/kg, 7×10¹¹ vg/kg, 6×10¹¹ vg/kg, 5×10¹¹ vg/kg, 4×10¹¹ vg/kg, 3×10¹¹ vg/kg, 2×10¹¹ vg/kg, 1×10¹¹ vg/kg, 9×10¹⁰ vg/kg, 8×10¹⁰ vg/kg, 7×10¹⁰ vg/kg, 6×10¹⁰ vg/kg, 5×10¹⁰ vg/kg, 4×10¹⁰ vg/kg, 3×10¹⁰ vg/kg, 2×10¹⁰ vg/kg, 1×10¹⁰ vg/kg, 9×10⁹ vg/kg, 8×10⁹ vg/kg, 7×10⁹ vg/kg, 6×10⁹ vg/kg, 5×10⁹ vg/kg, 4×10⁹ vg/kg, 3×10⁹ vg/kg, 2×10⁹ vg/kg, 1×10⁹ vg/kg, 9×10⁸ vg/kg, 8×10⁸ vg/kg, 7×10⁸ vg/kg, 6×10⁸ vg/kg, 5×10⁸ vg/kg, 4×10⁸ vg/kg, 3×10⁸ vg/kg, 2×10⁸ vg/kg, or 1×10⁸ vg/kg, or falls within a range defined by any two of these values. The above listed dosages being in vg/kg heart tissue units.

Apart from viral vectors, non-viral expression constructs may also be used for introducing a gene encoding a target protein or a functioning variant or fragment thereof into a cell of a patient. Non-viral expression vectors which permit the in vivo expression of protein in the target cell include, for example, a plasmid, a modified RNA, a cDNA, antisense oligomers, DNA-lipid complexes, nanoparticles, exosomes, any other non-viral shuttle suitable for gene therapy, variations thereof, and a combination thereof.

Apart from viral vectors and non-viral expression vectors, nuclease systems may also be used, in conjunction with a vector and/or an electroporation system, to enter into a cell of a patient and introduce therein a gene encoding a target protein or a functioning variant or fragment thereof. Exemplary nuclease systems may include, without limitations, a clustered regularly interspaced short palindromic repeats (CRISPR), a DNA cutting enzyme (e.g., Cas9), meganucleases, TALENs, zinc finger nucleases, any other nuclease system suitable for gene therapy, variations thereof, and a combination thereof. For instance, in one embodiment, one viral vector (e.g., AAV) may be used for a nuclease (e.g., CRISPR) and another viral vector (e.g., AAV) may be used for a DNA cutting enzyme (e.g., Cas9) to introduce both (the nuclease and the DNA cutting enzyme) into a target cell.

Other vector delivery systems which can be employed to deliver a therapeutic polynucleotide sequence encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific. Receptor-mediated gene targeting vehicles may include two components: a cell receptor-specific ligand and a DNA-binding agent.

Suitable methods for the transfer of non-viral vectors into target cells are, for example, the lipofection method, the calcium-phosphate co-precipitation method, the DEAE-dextran method and direct DNA introduction methods using micro-glass tubes, ultrasound, electroporation, and the like. Prior to the introduction of the vector, the cardiac muscle cells may be treated with a permeabilization agent, such as phosphatidylcholine, streptolysins, sodium caprate, decanoylcarnitine, tartaric acid, lysolecithin, Triton X-100, and the like. Exosomes may also be used to transfer naked DNA or AAV-encapsidated DNA.

A gene therapy vector of the invention may comprise a promoter that is functionally linked to the nucleic acid sequence encoding to the target protein. The promoter sequence must be compact and ensure a strong expression. Preferably, the promoter provides for an expression of the target protein in the myocardium of the patient that has been treated with the gene therapy vector. In some embodiment, the gene therapy vector comprises a cardiac-specific promoter which is operably linked to the nucleic acid sequence encoding the target protein. As used herein, a “cardiac-specific promoter” refers to a promoter whose activity in cardiac cells is at least 2-fold higher than in any other non-cardiac cell type. Preferably, a cardiac-specific promoter suitable for being used in the vector of the invention has an activity in cardiac cells which is at least 5-fold, at least 10-fold, at least 15-fold, at least 20-fold, at least 25-fold, or at least 50-fold higher compared to its activity in a non-cardiac cell type.

The cardiac-specific promoter may be a selected human promoter, or a promoter comprising a functionally equivalent sequence having at least about 80%, at least about 90%, at least about 95%, at least about 96%, at least about 97%, at least about 98%, or at least about 99% sequence identity to the selected human promoter. An exemplary non-limiting promoter that may be used is a cardiac troponin T promoter (TNNT2). Other non-limiting examples of promoters include alpha myosin heavy chain promoter, the myosin light chain 2v promoter, the alpha myosin heavy chain promoter, the alpha-cardiac actin promoter, the alpha-tropomyosin promoter, the cardiac troponin C promoter, the cardiac troponin I promoter, the cardiac myosin-binding protein C promoter, and the sarco/endoplasmic reticulum Ca²⁺-ATPase (SERCA) promoter (e.g., isoform 2 of this promoter (SERCA2)).

The vectors useful in the present invention may have varying transduction efficiencies. As a result, the viral or non-viral vector transduces more than, equal to, or at least about 10%, about 20%, about 30%, about 40%, about 50%, about 55%, about 60%, about 65%, about 70%, about 75%, about 80%, about 85%, about 90%, about 95%, about 99%, or 100% of the cells of the targeted vascular territory. More than one vector (viral or non-viral, or combinations thereof) can be used simultaneously or in sequence. This can be used to transfer more than one polynucleotide, and/or target more than one type of cell. Where multiple vectors or multiple agents are used, more than one transduction/transfection efficiency can result.

Pharmaceutical compositions that contain gene therapy vectors may be prepared either as liquid solutions or suspensions. The pharmaceutical composition of the invention can include commonly used pharmaceutically acceptable excipients, such as diluents and carriers. In particular, the composition comprises a pharmaceutically acceptable carrier, e.g., water, saline, Ringer's solution, or dextrose solution. In addition to the carrier, the pharmaceutical composition may also contain emulsifying agents, pH buffering agents, stabilizers, dyes, and the like.

In certain embodiments, a pharmaceutical composition will comprise a therapeutically effective gene dose, which is a dose that is capable of preventing or treating cardiomyopathy in a subject, without being toxic to the subject. Prevention or treatment of cardiomyopathy may be assessed as a change in a phenotypic characteristic associated with cardiomyopathy with such change being effective to prevent or treat cardiomyopathy. Thus, a therapeutically effective gene dose is typically one that, when administered in a physiologically tolerable composition, is sufficient to improve or prevent the pathogenic heart phenotype in the treated subject.

In certain embodiments, gene therapy vectors may be transduced into a subject through several different methods, including intravenous delivery, intraarterial delivery, or intraperitoneal delivery. In some embodiments, a gene therapy vector may be administered directly to heart tissue, for example, by intracoronary administration. In some embodiments, tissue transduction of the myocardium may be achieved by catheter-mediated intramyocardial delivery, which may be used to transfer vector-free cDNA coupled or uncoupled to transduction-enhancing carriers into myocardium.

In certain embodiments, the drug will comprise a therapeutically effective gene dose. A therapeutically effective gene dose is one that is capable of preventing or treating a particular heart condition in a patient, without being toxic to the patient.

Heart conditions that may be treated by the methods disclosed herein may include, without limitations, one or more of a genetically determined heart disease (e.g., genetically determined cardiomyopathy), arrhythmic heart disease, heart failure, ischemia, arrhythmia, myocardial infarction, congestive heart failure, transplant rejection, abnormal heart contractility, non-ischemic cardiomyopathy, mitral valve regurgitation, aortic stenosis or regurgitation, abnormal Ca²⁺ metabolism, congenital heart disease, primary or secondary cardiac tumors, and combinations thereof.

### ILLUSTRATIVE PROPHETIC EXAMPLES

The following example is set forth to assist in understanding the disclosure and should not, of course, be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

In an illustrative example of an in vitro system, a PKP2 isoform 2a cDNA sequence (2764bp cDNA, GenBank: BC126199.1; SEQ ID NO:1) is cloned under the cardiac-specific TNNT2 promoter (SEQ ID NO:5) and using AAV2 internal terminal repeats (ITRs): ITR-TNNT2-PKP2cDNA-ITR. As another illustrative example, the nucleic acid sequence encoding PKP2 may be a codon-optimized version of the PKP2 gene (SEQ ID NO:3) encoding for PKP2 isoform 2b (SEQ ID NO: 4). The construct may be vectorized into AAV, such as AAV6 and AAV9. A construct with Flag added on (Flag-PKP2) may be prepared in order to be able to identify protein after transfection by anti-Flag and distinguish it from endogenous protein. SEQ ID NO:6 is an exemplary construct sequence for expressing, for example, PKP2 isoform 2b. Expression of PKP2 in vitro may be observed with immunofluorescence microscopy using PKP2 primary antibodies, which reveals localization of the PKP2 at the cell membranes and in dense plaques.

The AAV6-TNNT2-PKP2 is used to transfect iPSC-CMs in 2D cell cultures including: normal cardiomyocytes; cardiomyocytes carrying 1 heterozygous PKP2 mutation (from ARVC patients); and cardiomyocytes carrying two PKP2 mutations in trans.

After successful transfection and characterization of PKP2 RNA and protein levels, a comparison of normal versus PKP2-deficient and PKP2-corrected CM is performed for a number of readouts, including: cell-size and correction of genes with known altered expression in PKP2 deficiency (MYL2, SCN5A (whose protein product is NaV1.5), GJA1, and TTN).

It is contemplated that similar methodologies may be adapted for ex vivo treatment in a human 3D culture model as well as in vivo treatment in a PKP2-mutated mouse model.

It is believed that when (Flag-)PKP2 protein gets expressed, it will arrive at its correct sub cellular localization (the desmosome), and that transfection corrects PKP2-haploinsufficient or completely deficient cells at the RNA level and at the protein level. In completely PKP2-deficient cells, it is believed PKP2-transfection is also capable of restoring the desmosomal protein complex in the desmosome, in particular the restoration of plakoglobin, which is reduced when PKP2 is diminished.

It is further contemplated that the gene therapy vector for expressing PKP2 isoform 2a, PKP2 isoform 2b, or both may be delivered to cardiac tissue of a human subject. For example, the gene therapy vector may be formulated into a therapeutic formulation that includes one or more gene therapy vectors and a pharmaceutically acceptable excipient or carrier. The formulation may be transduced into the human subject through several different methods, including intravenous delivery, intraarterial delivery, or intraperitoneal delivery. The gene therapy vector may be administered directly to heart tissue, for example, by intracoronary administration. The gene therapy vector may also be delivered via catheter-mediated intramyocardial delivery.

It is further contemplated that the gene therapy vector may be administered locally to the subject's heart tissue, for example, by isolating the subject's coronary circulation from the subject's systemic circulation thus forming a closed circuit, and perfusing a fluid (e.g., a formulation comprising the gene therapy vector) into the subject's isolated coronary circulation. The perfusion may be performed in the subject's unarrested beating heart. The closed circuit may be formed, for example, with a first drug delivery catheter positioned in the patient's right coronary artery, a second drug delivery catheter positioned in a patient's left main coronary artery, a drug collection catheter positioned in a coronary sinus, the coronary artery, the coronary venous system, and an external membrane oxygenator interspersed between the venous and arterial branches. Such local delivery may be performed as described with respect to International Application No. PCT/IB2020/000692, filed Aug. 26, 2020, the disclosure of which is hereby incorporated by reference herein in its entirety.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

SEQ ID NO:1 below is a cDNA copy of an mRNA sequence that includes a protein coding sequence for PKP2 isoform 2a (GenBank: BC126199.1):

GAGTCCAGAGGCAGGCGAGCAGCTCGGTCGCCCCCACCGGCCCCATGGCA
GCCCCCGGCGCCCCAGCTGAGTACGGCTACATCCGGACCGTCCTGGGCCA
GCAGATCCTGGGACAACTGGACAGCTCCAGCCTGGCGCTGCCCTCCGAGG
CCAAGCTGAAGCTGGCGGGGAGCAGCGGCCGCGGCGGCCAGACAGTCAAG
AGCCTGCGGATCCAGGAGCAGGTGCAGCAGACCCTCGCCCGGAAGGGCCG
CAGCTCCGTGGGCAACGGAAATCTTCACCGAACCAGCAGTGTTCCTGAGT
ATGTCTACAACCTACACTTGGTTGAAAATGATTTTGTTGGAGGCCGTTCC
CCTGTTCCTAAAACCTATGACATGCTAAAGGCTGGCACAACTGCCACTTA
TGAAGGTCGCTGGGGAAGAGGAACAGCACAGTACAGCTCCCAGAAGTCCG
TGGAAGAAAGGTCCTTGAGGCATCCTCTGAGGAGACTGGAGATTTCTCCT
GACAGCAGCCCGGAGAGGGCTCACTACACGCACAGCGATTACCAGTACAG
CCAGAGAAGCCAGGCTGGGCACACCCTGCACCACCAAGAAAGCAGGCGGG
CCGCCCTCCTAGTGCCACCGAGATATGCTCGTTCCGAGATCGTGGGGGTC
AGCCGTGCTGGCACCACAAGCAGGCAGCGCCACTTTGACACATACCACAG
ACAGTACCAGCATGGCTCTGTTAGCGACACCGTTTTTGACAGCATCCCTG
CCAACCCGGCCCTGCTCACGTACCCCAGGCCAGGGACCAGCCGCAGCATG
GGCAACCTCTTGGAGAAGGAGAACTACCTGACGGCAGGGCTCACTGTCGG
GCAGGTCAGGCCGCTGGTGCCCCTGCAGCCCGTCACTCAGAACAGGGCTT
CCAGGTCCTCCTGGCATCAGAGCTCCTTCCACAGCACCCGCACGCTGAGG
GAAGCTGGGCCCAGTGTCGCCGTGGATTCCAGCGGGAGGAGAGCGCACTT
GACTGTCGGCCAGGCGGCCGCAGGGGGAAGTGGGAATCTGCTCACTGAGA
GAAGCACTTTCACTGACTCCCAGCTGGGGAATGCAGACATGGAGATGACT
CTGGAGCGAGCAGTGAGTATGCTCGAGGCAGACCACATGCTGCCATCCAG
GATTTCTGCTGCAGCTACTTTCATACAGCACGAGTGCTTCCAGAAATCTG
AAGCTCGGAAGAGGGTTAACCAGCTTCGTGGCATCCTCAAGCTTCTGCAG
CTCCTAAAAGTTCAGAATGAAGACGTTCAGCGAGCTGTGTGTGGGGCCTT
GAGAAACTTAGTATTTGAAGACAATGACAACAAATTGGAGGTGGCTGAAC
TAAATGGGGTACCTCGGCTGCTCCAGGTGCTGAAGCAAACCAGAGACTTG
GAGACTAAAAAACAAATAACAGGTTTGCTGTGGAATTTGTCATCTAATGA
CAAACTCAAGAATCTCATGATAACAGAAGCATTGCTTACGCTGACGGAGA
ATATCATCATCCCCTTTTCTGGGTGGCCTGAAGGAGACTACCCAAAAGCA
AATGGTTTGCTCGATTTTGACATATTCTACAACGTCACTGGATGCCTAAG
AAACATGAGTTCTGCTGGCGCTGATGGGAGAAAAGCGATGAGAAGATGTG
ACGGACTCATTGACTCACTGGTCCATTATGTCAGAGGAACCATTGCAGAT
TACCAGCCAGATGACAAGGCCACGGAGAATTGTGTGTGCATTCTTCATAA
CCTCTCCTACCAGCTGGAGGCAGAGCTCCCAGAGAAATATTCCCAGAATA
TCTATATTCAAAACCGGAATATCCAGACTGACAACAACAAAAGTATTGGA
TGTTTTGGCAGTCGAAGCAGGAAAGTAAAAGAGCAATACCAGGACGTGCC
GATGCCGGAGGAAAAGAGCAACCCCAAGGGCGTGGAGTGGCTGTGGCATT
CCATTGTTATAAGGATGTATCTGTCCTTGATCGCCAAAAGTGTCCGCAAC
TACACACAAGAAGCATCCTTAGGAGCTCTGCAGAACCTCACGGCCGGAAG
TGGACCAATGCCGACATCAGTGGCTCAGACAGTTGTCCAGAAGGAAAGTG
GCCTGCAGCACACCCGAAAGATGCTGCATGTTGGTGACCCAAGTGTGAAA
AAGACAGCCATCTCGCTGCTGAGGAATCTGTCCCGGAATCTTTCTCTGCA
GAATGAAATTGCCAAAGAAACTCTCCCTGATTTGGTTTCCATCATTCCTG
ACACAGTCCCGAGTACTGACCTTCTCATTGAAACTACAGCCTCTGCCTGT
TACACATTGAACAACATAATCCAAAACAGTTACCAGAATGCACGCGACCT
TCTAAACACCGGGGGCATCCAGAAAATTATGGCCATTAGTGCAGGCGATG
CCTATGCCTCCAACAAAGCAAGTAAAGCTGCTTCCGTCCTTCTGTATTCT
CTGTGGGCACACACGGAACTGCATCATGCCTACAAGAAGGCTCAGTTTAA
GAAGACAGATTTTGTCAACAGCCGGACTGCCAAAGCCTACCACTCCCTTA
AAGACTGAGGAAAATGACAAAGTATTCTCGGCTGCAAAAATCCCCAAAGG
AAAACACCTATTTTTCTACTACCCAGCCCAAGAAACCTCAAAAGCATGCC
TTGTTTCTATCCTTCTCTATTTCCGTGGTCCCCTGAATCCAGAAAACAAA
TAGAACATAATTTTATGAGTCTTCCAGAAGACCTTTGCAAGTTTGCCACC
AGTAGATACCGGCC

SEQ ID NO:2 below is the amino acid sequence for PKP2 isoform 2a:

MAAPGAPAEYGYIRTVLGQQILGQLDSSSLALPSEAKLKLAGSSGRGGQT
VKSLRIQEQVQQTLARKGRSSVGNGNLHRTSSVPEYVYNLHLVENDFVGG
RSPVPKTYDMLKAGTTATYEGRWGRGTAQYSSQKSVEERSLRHPLRRLEI
SPDSSPERAHYTHSDYQYSQRSQAGHTLHHQESRRAALLVPPRYARSEIV
GVSRAGTTSRQRHFDTYHRQYQHGSVSDTVFDSIPANPALLTYPRPGTSR
SMGNLLEKENYLTAGLTVGQVRPLVPLQPVTQNRASRSSWHQSSFHSTRT
LREAGPSVAVDSSGRRAHLTVGQAAAGGSGNLLTERSTFTDSQLGNADME
MTLERAVSMLEADHMLPSRISAAATFIQHECFQKSEARKRVNQLRGILKL
LQLLKVQNEDVQRAVCGALRNLVFEDNDNKLEVAELNGVPRLLQVLKQTR
DLETKKQITGLLWNLSSNDKLKNLMITEALLTLTENIIIPFSGWPEGDYP
KANGLLDFDIFYNVTGCLRNMSSAGADGRKAMRRCDGLIDSLVHYVRGTI
ADYQPDDKATENCVCILHNLSYQLEAELPEKYSQNIYIQNRNIQTDNNKS
IGCFGSRSRKVKEQYQDVPMPEEKSNPKGVEWLWHSIVIRMYLSLIAKSV
RNYTQEASLGALQNLTAGSGPMPTSVAQTVVQKESGLQHTRKMLHVGDPS
VKKTAISLLRNLSRNLSLQNEIAKETLPDLVSIIPDTVPSTDLLIETTAS
ACYTLNNIIQNSYQNARDLLNTGGIQKIMAISAGDAYASNKASKAASVLL
YSLWAHTELHHAYKKAQFKKTDFVNSRTAKAYHSLKD

SEQ ID NO:3 below is a codon-optimized cDNA sequence encoding for PKP2 isoform 2b:

ATGGCCGCCCCCGGAGCACCTGCCGAGTATGGCTACATTCGCACCGTCCT
GGGACAGCAGATTCTGGGACAGCTGGATTCATCAAGCCTGGCCCTGCCTT
CTGAGGCCAAGCTGAAGCTGGCAGGAAGCTCCGGAAGGGGAGGACAGACC
GTGAAGAGCCTGAGAATCCAGGAGCAGGTGCAGCAGACACTGGCCCGGAA
GGGCAGATCTAGCGTGGGCAACGGCAATCTGCACAGGACCTCCTCTGTGC
CAGAGTACGTGTATAACCTGCACCTGGTGGAGAATGACTTCGTGGGAGGC
CGCAGCCCAGTGCCAAAGACATACGATATGCTGAAGGCCGGCACCACAGC
AACCTATGAGGGCAGGTGGGGAAGAGGAACAGCACAGTACAGCTCCCAGA
AGTCTGTGGAGGAGCGGAGCCTGAGACACCCTCTGCGGAGACTGGAGATC
AGCCCAGACTCTAGCCCTGAGAGGGCACACTATACCCACTCCGATTACCA
GTATTCTCAGAGAAGCCAGGCAGGACACACACTGCACCACCAGGAGAGCA
GGAGGGCCGCCCTGCTGGTGCCACCTAGATACGCCCGCTCTGAGATCGTG
GGCGTGAGCAGGGCAGGAACCACATCCCGGCAGAGACACTTCGACACCTA
CCACAGACAGTATCAGCACGGCTCTGTGAGCGACACAGTGTTTGATTCCA
TCCCTGCCAACCCAGCCCTGCTGACCTATCCTCGGCCAGGCACATCCAGA
TCTATGGGCAACCTGCTGGAGAAGGAGAATTACCTGACCGCAGGCCTGAC
AGTGGGACAGGTGAGGCCCCTGGTGCCTCTGCAGCCAGTGACCCAGAATC
GGGCCAGCAGATCCTCTTGGCACCAGAGCTCCTTCCACTCTACCAGGACA
CTGAGGGAGGCAGGACCAAGCGTGGCAGTGGACTCTAGCGGCCGGAGAGC
CCACCTGACCGTGGGACAGGCAGCAGCAGGAGGATCCGGCAACCTGCTGA
CAGAGAGGTCCACCTTTACAGACTCTCAGCTGGGCAATGCCGATATGGAG
ATGACCCTGGAGAGGGCCGTGAGCATGCTGGAGGCAGACCACATGCTGCC
ATCCAGGATCTCTGCCGCAGCCACATTCATCCAGCACGAGTGCTTTCAGA
AGTCCGAGGCAAGGAAGAGGGTGAACCAGCTGAGGGGCATCCTGAAGCTG
CTGCAGCTGCTGAAGGTGCAGAACGAGGATGTGCAGAGGGCCGTGTGCGG
CGCCCTGAGGAATCTGGTGTTCGAGGACAACGATAATAAGCTGGAGGTGG
CAGAGCTGAACGGAGTGCCAAGGCTGCTGCAGGTGCTGAAGCAGACCCGC
GACCTGGAGACAAAGAAGCAGATCACCGATCACACAGTGAACCTGCGGAG
CAGAAATGGATGGCCTGGAGCAGTGGCACACGCATGCAATCCAAGCACCC
TGGGAGGACAGGGAGGAAGGATCACAAGATCCGGCGTGCGGGACCAGCCT
GATCAGCACGGCCTGCTGTGGAACCTGTCCTCTAATGACAAGCTGAAGAA
CCTGATGATCACCGAGGCCCTGCTGACCCTGACAGAGAATATCATCATCC
CTTTTAGCGGCTGGCCAGAGGGCGATTATCCCAAGGCCAACGGCCTGCTG
GACTTCGATATCTTTTACAACGTGACCGGCTGCCTGAGGAATATGAGCTC
CGCCGGAGCAGACGGAAGAAAGGCCATGAGGCGCTGTGACGGCCTGATCG
ATTCCCTGGTGCACTACGTGCGGGGCACCATCGCCGATTATCAGCCCGAC
GATAAGGCCACAGAGAACTGCGTGTGCATCCTGCACAATCTGTCTTATCA
GCTGGAGGCCGAGCTGCCTGAGAAGTACAGCCAGAACATCTATATCCAGA
ACAGAAATATCCAGACCGACAACAATAAGAGCATCGGCTGCTTCGGCAGC
AGGTCCCGCAAGGTGAAGGAGCAGTACCAGGATGTGCCCATGCCTGAGGA
GAAGTCCAATCCCAAGGGCGTGGAGTGGCTGTGGCACTCTATCGTGATCA
GGATGTATCTGAGCCTGATCGCCAAGTCCGTGCGCAACTACACCCAGGAG
GCATCTCTGGGCGCCCTGCAGAATCTGACAGCAGGATCTGGACCAATGCC
CACCAGCGTGGCCCAGACAGTGGTGCAGAAGGAGTCCGGCCTGCAGCACA
CCCGGAAGATGCTGCACGTGGGCGACCCATCCGTGAAGAAGACAGCCATC
TCTCTGCTGAGGAACCTGAGCCGCAATCTGTCCCTGCAGAACGAGATCGC
CAAGGAGACACTGCCCGATCTGGTGAGCATCATCCCAGACACCGTGCCCT
CCACAGATCTGCTGATCGAGACAACAGCCTCCGCCTGTTACACCCTGAAC
AATATCATCCAGAACTCTTATCAGAATGCCCGGGACCTGCTGAACACAGG
CGGCATCCAGAAGATCATGGCAATCTCCGCCGGCGATGCATACGCATCTA
ATAAGGCCAGCAAGGCCGCCTCCGTGCTGCTGTATTCTCTGTGGGCACAC
ACCGAGCTGCACCACGCATACAAGAAGGCCCAGTTTAAGAAGACTGATTT
CGTGAATAGCAGAACAGCCAAAGCCTACCACAGCCTGAAGGAC

SEQ ID NO: 4 below is the amino acid sequence for PKP2 isoform 2b:

MAAPGAPAEYGYIRTVLGQQILGQLDSSSLALPSEAKLKLAGSSGRGGQT
VKSLRIQEQVQQTLARKGRSSVGNGNLHRTSSVPEYVYNLHLVENDFVGG
RSPVPKTYDMLKAGTTATYEGRWGRGTAQYSSQKSVEERSLRHPLRRLEI
SPDSSPERAHYTHSDYQYSQRSQAGHTLHHQESRRAALLVPPRYARSEIV
GVSRAGTTSRQRHFDTYHRQYQHGSVSDTVFDSIPANPALLTYPRPGTSR
SMGNLLEKENYLTAGLTVGQVRPLVPLQPVTQNRASRSSWHQSSFHSTRT
LREAGPSVAVDSSGRRAHLTVGQAAAGGSGNLLTERSTFTDSQLGNADME
MTLERAVSMLEADHMLPSRISAAATFIQHECFQKSEARKRVNQLRGILKL
LQLLKVQNEDVQRAVCGALRNLVFEDNDNKLEVAELNGVPRLLQVLKQTR
DLETKKQITDHTVNLRSRNGWPGAVAHACNPSTLGGQGGRITRSGVRDQP
DQHGLLWNLSSNDKLKNLMITEALLTLTENIIIPFSGWPEGDYPKANGLL
DFDIFYNVTGCLRNMSSAGADGRKAMRRCDGLIDSLVHYVRGTIADYQPD
DKATENCVCILHNLSYQLEAELPEKYSQNIYIQNRNIQTDNNKSIGCFGS
RSRKVKEQYQDVPMPEEKSNPKGVEWLWHSIVIRMYLSLIAKSVRNYTQE
ASLGALQNLTAGSGPMPTSVAQTVVQKESGLQHTRKMLHVGDPSVKKTAI
SLLRNLSRNLSLQNEIAKETLPDLVSIIPDTVPSTDLLIETTASACYTLN
NIIQNSYQNARDLLNTGGIQKIMAISAGDAYASNKASKAASVLLYSLWAH
TELHHAYKKAQFKKTDFVNSRTAKAYHSLKD

SEQ ID NO:5 below is a nucleic acid sequence encoding a TNNT2 promoter:

GTCATGGAGAAGACCCACCTTGCAGATGTCCTCACTGGGGCTGGCAGAGC
CGGCAACCTGCCTAAGGCTGCTCAGTCCATTAGGAGCCAGTAGCCTGGAA
GATGTCTTTACCCCCAGCATCAGTTCAAGTGGAGCAGCACATAACTCTTG
CCCTCTGCCTTCCAAGATTCTGGTGCTGAGACTTATGGAGTGTCTTGGAG
GTTGCCTTCTGCCCCCCAACCCTGCTCCCAGCTGGCCCTCCCAGGCCTGG
GTTGCTGGCCTCTGCTTTATCAGGATTCTCAAGAGGGACAGCTGGTTTAT
GTTGCATGACTGTTCCCTGCATATCTGCTCTGGTTTTAAATAGCTTATCT
GAGCAGCTGGAGGACCACATGGGCTTATATGGCGTGGGGTACATGTTCCT
GTAGCCTTGTCCCTGGCACCTGCCAAAATAGCAGCCAACACCCCCCACCC
CCACCGCCATCCCCCTGCCCCACCCGTCCCCTGTCGCACATTCCTCCCTC
CGCAGGGCTGGCTCACCAGGCCCCAGCCCACATGCCTGCTTAAAGCCCTC
TCCATCCTCTGCCTCACCCAGTCCCCGCTGAGACTGAGCAGACGCCTCCA

SEQ ID NO:6 below is an exemplary vector construct for expressing PKP2 isoform 2b in a cardiomyocyte:

GGCACTGGGCAGGTAAGTATCAAGGTTACAAGACAGGTTTAAGGAGACCA
ATAGAAACTGGGCTTGTCGAGACAGAGAAGACTCTTGCGTTTCTGATAGG
CACCTATTGGTCTTACTGACATCCACTTTGCCTTTCTCTCCACAGGTGTC
CACTCCCAGTTCAATTACAGCTCTTAAGGCTAGAGTACTTAATACGACTC
ACTATAGGCTAGCGGTACCGGTCGCCACCATGGACTACAAAGACCATGAC
GGTGATTATAAAGATCATGACATCGATTACAAGGATGACGATGACAAGCT
TGGTACCGAGCTCGGATCCATGGCCGCCCCCGGAGCACCTGCCGAGTATG
GCTACATTCGCACCGTCCTGGGACAGCAGATTCTGGGACAGCTGGATTCA
TCAAGCCTGGCCCTGCCTTCTGAGGCCAAGCTGAAGCTGGCAGGAAGCTC
CGGAAGGGGAGGACAGACCGTGAAGAGCCTGAGAATCCAGGAGCAGGTGC
AGCAGACACTGGCCCGGAAGGGCAGATCTAGCGTGGGCAACGGCAATCTG
CACAGGACCTCCTCTGTGCCAGAGTACGTGTATAACCTGCACCTGGTGGA
GAATGACTTCGTGGGAGGCCGCAGCCCAGTGCCAAAGACATACGATATGC
TGAAGGCCGGCACCACAGCAACCTATGAGGGCAGGTGGGGAAGAGGAACA
GCACAGTACAGCTCCCAGAAGTCTGTGGAGGAGCGGAGCCTGAGACACCC
TCTGCGGAGACTGGAGATCAGCCCAGACTCTAGCCCTGAGAGGGCACACT
ATACCCACTCCGATTACCAGTATTCTCAGAGAAGCCAGGCAGGACACACA
CTGCACCACCAGGAGAGCAGGAGGGCCGCCCTGCTGGTGCCACCTAGATA
CGCCCGCTCTGAGATCGTGGGCGTGAGCAGGGCAGGAACCACATCCCGGC
AGAGACACTTCGACACCTACCACAGACAGTATCAGCACGGCTCTGTGAGC
GACACAGTGTTTGATTCCATCCCTGCCAACCCAGCCCTGCTGACCTATCC
TCGGCCAGGCACATCCAGATCTATGGGCAACCTGCTGGAGAAGGAGAATT
ACCTGACCGCAGGCCTGACAGTGGGACAGGTGAGGCCCCTGGTGCCTCTG
CAGCCAGTGACCCAGAATCGGGCCAGCAGATCCTCTTGGCACCAGAGCTC
CTTCCACTCTACCAGGACACTGAGGGAGGCAGGACCAAGCGTGGCAGTGG
ACTCTAGCGGCCGGAGAGCCCACCTGACCGTGGGACAGGCAGCAGCAGGA
GGATCCGGCAACCTGCTGACAGAGAGGTCCACCTTTACAGACTCTCAGCT
GGGCAATGCCGATATGGAGATGACCCTGGAGAGGGCCGTGAGCATGCTGG
AGGCAGACCACATGCTGCCATCCAGGATCTCTGCCGCAGCCACATTCATC
CAGCACGAGTGCTTTCAGAAGTCCGAGGCAAGGAAGAGGGTGAACCAGCT
GAGGGGCATCCTGAAGCTGCTGCAGCTGCTGAAGGTGCAGAACGAGGATG
TGCAGAGGGCCGTGTGCGGCGCCCTGAGGAATCTGGTGTTCGAGGACAAC
GATAATAAGCTGGAGGTGGCAGAGCTGAACGGAGTGCCAAGGCTGCTGCA
GGTGCTGAAGCAGACCCGCGACCTGGAGACAAAGAAGCAGATCACCGATC
ACACAGTGAACCTGCGGAGCAGAAATGGATGGCCTGGAGCAGTGGCACAC
GCATGCAATCCAAGCACCCTGGGAGGACAGGGAGGAAGGATCACAAGATC
CGGCGTGCGGGACCAGCCTGATCAGCACGGCCTGCTGTGGAACCTGTCCT
CTAATGACAAGCTGAAGAACCTGATGATCACCGAGGCCCTGCTGACCCTG
ACAGAGAATATCATCATCCCTTTTAGCGGCTGGCCAGAGGGCGATTATCC
CAAGGCCAACGGCCTGCTGGACTTCGATATCTTTTACAACGTGACCGGCT
GCCTGAGGAATATGAGCTCCGCCGGAGCAGACGGAAGAAAGGCCATGAGG
CGCTGTGACGGCCTGATCGATTCCCTGGTGCACTACGTGCGGGGCACCAT
CGCCGATTATCAGCCCGACGATAAGGCCACAGAGAACTGCGTGTGCATCC
TGCACAATCTGTCTTATCAGCTGGAGGCCGAGCTGCCTGAGAAGTACAGC
CAGAACATCTATATCCAGAACAGAAATATCCAGACCGACAACAATAAGAG
CATCGGCTGCTTCGGCAGCAGGTCCCGCAAGGTGAAGGAGCAGTACCAGG
ATGTGCCCATGCCTGAGGAGAAGTCCAATCCCAAGGGCGTGGAGTGGCTG
TGGCACTCTATCGTGATCAGGATGTATCTGAGCCTGATCGCCAAGTCCGT
GCGCAACTACACCCAGGAGGCATCTCTGGGCGCCCTGCAGAATCTGACAG
CAGGATCTGGACCAATGCCCACCAGCGTGGCCCAGACAGTGGTGCAGAAG
GAGTCCGGCCTGCAGCACACCCGGAAGATGCTGCACGTGGGCGACCCATC
CGTGAAGAAGACAGCCATCTCTCTGCTGAGGAACCTGAGCCGCAATCTGT
CCCTGCAGAACGAGATCGCCAAGGAGACACTGCCCGATCTGGTGAGCATC
ATCCCAGACACCGTGCCCTCCACAGATCTGCTGATCGAGACAACAGCCTC
CGCCTGTTACACCCTGAACAATATCATCCAGAACTCTTATCAGAATGCCC
GGGACCTGCTGAACACAGGCGGCATCCAGAAGATCATGGCAATCTCCGCC
GGCGATGCATACGCATCTAATAAGGCCAGCAAGGCCGCCTCCGTGCTGCT
GTATTCTCTGTGGGCACACACCGAGCTGCACCACGCATACAAGAAGGCCC
AGTTTAAGAAGACTGATTTCGTGAATAGCAGAACAGCCAAAGCCTACCAC
AGCCTGAAGGACCTCGAGGGATCTGGAGCAACAAACTTCTCACTACTCAA
ACAAGCAGGTGACGTGGAGGAGAATCCCGGGCCTAAGCTTATGAAAACCT
TCAACATCTCTCAGCAGGATCTGGAGCTGGTGGAGGTCGCCACTGAGAAG
ATCACCATGCTCTATGAGGACAACAAGCACCATGTCGGGGCGGCCATCAG
GACCAAGACTGGGGAGATCATCTCTGCTGTCCACATTGAAGCCTACATTG
GCAGGGTCACTGTCTGTGCTGAAGCCATTGCCATTGGGTCTGCTGTGAGC
AACGGGCAGAAGGACTTTGACACCATTGTGGCTGTCAGGCACCCCTACTC
TGATGAGGTGGACAGATCCATCAGGGTGGTCAGCCCCTGTGGCATGTGTA
GAGAGCTGATCTCTGACTATGCTCCTGACTGCTTTGTGCTCATTGAGATG
AATGGCAAGCTGGTCAAAACCACCATTGAGGAACTCATCCCCCTCAAGTA
CACCAGGAACTAATAAGCGGCCGCTTCCCTTTAGTGAGGGTTAATGCTTC
GAGCAGACATGATAAGATACATTGATGAGTTTGGACAAACCACAACTAGA
ATGCAGTGAAAAAAATGCTTTATTTGTGAAATTTGTGATGCTATTGCTTT
ATTTGTAACCATTATAAGCTGCAATAAACAAGTTAACAACAACAATTGCA
TTCATTTTATGTTTCAGGTTCAGGGGGAGATGTGGGAGGTTTTTTAAAGC
AAGTAAAACCTCTACAAATGTGGTAAAATCCGATAAGGGACTAGAGCATG
GCTACGTAGATAAGTAGCATGGCGGGTTAATCATTAACTACAAGGAACCC
CTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCT
CAGTGAGCGAGCGAGCGCGCCAGCTGGCGTAATAGCGAAGAGGCCCGCAC
CGATCGCCCTTCCCAACAGTTGCGCAGCCTGAATGGCGAATGGAATTCCA
GACGATTGAGCGTCAAAATGTAGGTATTTCCATGAGCGTTTTTCCGTTGC
AATGGCTGGCGGTAATATTGTTCTGGATATTACCAGCAAGGCCGATAGTT
TGAGTTCTTCTACTCAGGCAAGTGATGTTATTACTAATCAAAGAAGTATT
GCGACAACGGTTAATTTGCGTGATGGACAGACTCTTTTACTCGGTGGCCT
CACTGATTATAAAAACACTTCTCAGGATTCTGGCGTACCGTTCCTGTCTA
AAATCCCTTTAATCGGCCTCCTGTTTAGCTCCCGCTCTGATTCTAACGAG
GAAAGCACGTTATACGTGCTCGTCAAAGCAACCATAGTACGCGCCCTGTA
GCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCC
CTTTAGGGTTCCGATTTAGTGCTTTACGGCACCTCGACCCCAAAAAACTT
GATTAGGGTGATGGTTCACGTAGTGGGCCATCGCCCTGATAGACGGTTTT
TCGCCCTTTGACGTTGGAGTCCACGTTCTTTAATAGTGGACTCTTGTTCC
AAACTGGAACAACACTCAACCCTATCTCGGTCTATTCTTTTGATTTATAA
GGGATTTTGCCGATTTCGGCCTATTGGTTAAAAAATGAGCTGATTTAACA
AAAATTTAACGCGAATTTTAACAAAATATTAACGTCTACAATTTAAATAT
TTGCTTATACAATCTTCCTGTTTTTGGGGCTTTTCTGATTATCAACCGGG
GTACATATGATTGACATGCTAGTTTTACGATTACCGTTCATCGATTCTCT
TGTTTGCTCCAGACTCTCAGGCAATGACCTGATAGCCTTTGTAGAGACCT
CTCAAAAATAGCTACCCTCTCCGGCATGAATTTATCAGCTAGAACGGTTG
AATATCATATTGATGGTGATTTGACTGTCTCCGGCCTTTCTCACCCGTTT
GAATCTTTACCTACACATTACTCAGGCATTGCATTTAAAATATATGAGGG
TTCTAAAAATTTTTATCCTTGCGTTGAAATAAAGGCTTCTCCCGCAAAAG
TATTACAGGGTCATAATGTTTTTGGTACAACCGATTTAGCTTTATGCTCT
GAGGCTTTATTGCTTAATTTTGCTAATTCTTTGCCTTGCCTGTATGATTT
ATTGGATGTTGGAATCGCCTGATGCGGTATTTTCTCCTTACGCATCTGTG
CGGTATTTCACACCGCATATGGTGCACTCTCAGTACAATCTGCTCTGATG
CCGCATAGTTAAGCCAGCCCCGACACCCGCCAACACCCGCTGACGCGCCC
TGACGGGCTTGTCTGCTCCCGGCATCCGCTTACAGACAAGCTGTGACCGT
CTCCGGGAGCTGCATGTGTCAGAGGTTTTCACCGTCATCACCGAAACGCG
CGAGACGAAAGGGCCTCGTGATACGCCTATTTTTATAGGTTAATGTCATG
ATAATAATGGTTTCTTAGACGTCAGGTGGCACTTTTCGGGGAAATGTGCG
CGGAACCCCTATTTGTTTATTTTTCTAAATACATTCAAATATGTATCCGC
TCATGAGACAATAACCCTGATAAATGCTTCAATAATATTGAAAAAGGAAG
AGTATGAGTATTCAACATTTCCGTGTCGCCCTTATTCCCTTTTTTGCGGC
ATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTC
AACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTG
ACGCCGGGCAAGAGCAACTCGGTCGCCGCATACACTATTCTCAGAATGAC
TTGGTTGAGTACTCACCAGTCACAGAAAAGCATCTTACGGATGGCATGAC
AGTAAGAGAATTATGCAGTGCTGCCATAACCATGAGTGATAACACTGCGG
CCAACTTACTTCTGACAACGATCGGAGGACCGAAGGAGCTAACCGCTTTT
TTGCACAACATGGGGGATCATGTAACTCGCCTTGATCGTTGGGAACCGGA
GCTGAATGAAGCCATACCAAACGACGAGCGTGACACCACGATGCCTGTAG
CAATGGCAACAACGTTGCGCAAACTATTAACTGGCGAACTACTTACTCTA
GCTTCCCGGCAACAATTAATAGACTGGATGGAGGCGGATAAAGTTGCAGG
ACCACTTCTGCGCTCGGCCCTTCCGGCTGGCTGGTTTATTGCTGATAAAT
CTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGC
AACTATGGATGAACGAAATAGACAGATCGCTGAGATAGGTGCCTCACTGA
TTAAGCATTGGTAACTGTCAGACCAAGTTTACTCATATATACTTTAGATT
GATTTAAAACTTCATTTTTAATTTAAAAGGATCTAGGTGAAGATCCTTTT
TGATAATCTCATGACCAAAATCCCTTAACGTGAGTTTTCGTTCCACTGAG
CGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTG
GCTTCAGCAGAGCGCAGATACCAAATACTGTCCTTCTAGTGTAGCCGTAG
TTAGGCCACCACTTCAAGAACTCTGTAGCACCGCCTACATACCTCGCTCT
GCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGC
TGAACGGGGGGTTCGTGCACACAGCCCAGCTTGGAGCGAACGACCTACAC
CGAACTGAGATACCTACAGCGTGAGCTATGAGAAAGCGCCACGCTTCCCG
AAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGG
TTCCTGGCCTTTTGCTGGCCTTTTGCTCACATGTTCTTTCCTGCGTTATC
CCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCA
TTAATGCAGCTGCGCGCTCGCTCGCTCACTGAGGCCGCCCGGGCAAAGCC
CGGGCGTCGGGCGACCTTTGGTCGCCCGGCCTCAGTGAGCGAGCGAGCGC
GCAGAGAGGGAGTGGCCAACTCCATCACTAGGGGTTCCTTGTAGTTAATG
ATTAACCCGCCATGCTACTTATCTACGTAGCCATGCTCTAGATGTCATGG
AGAAGACCCACCTTGCAGATGTCCTCACTGGGGCTGGCAGAGCCGGCAAC
CTGCCTAAGGCTGCTCAGTCCATTAGGAGCCAGTAGCCTGGAAGATGTCT
TTACCCCCAGCATCAGTTCAAGTGGAGCAGCACATAACTCTTGCCCTCTG
CCTTCCAAGATTCTGGTGCTGAGACTTATGGAGTGTCTTGGAGGTTGCCT
TCTGCCCCCCAACCCTGCTCCCAGCTGGCCCTCCCAGGCCTGGGTTGCTG
GCCTCTGCTTTATCAGGATTCTCAAGAGGGACAGCTGGTTTATGTTGCAT
GACTGTTCCCTGCATATCTGCTCTGGTTTTAAATAGCTTATCTGAGCAGC
TGGAGGACCACATGGGCTTATATGGCGTGGGGTACATGTTCCTGTAGCCT
TGTCCCTGGCACCTGCCAAAATAGCAGCCAACACCCCCCACCCCCACCGC
CATCCCCCTGCCCCACCCGTCCCCTGTCGCACATTCCTCCCTCCGCAGGG
CTGGCTCACCAGGCCCCAGCCCACATGCCTGCTTAAAGCCCTCTCCATCC
TCTGCCTCACCCAGTCCCCGCTGAGACTGAGCAGACGCCTCCAGAGCTCG
GATCCTGAGAACTTCAGGGTGAGTCTATGGGAC

1. A method of treating or preventing cardiomyopathy in a human subject, the method comprising delivering a therapeutic dose of a gene therapy vector to cardiomyocytes of the human subject, wherein the gene therapy vector comprises a nucleic acid sequence encoding for plakophilin-2 (PKP2) or a functional variant thereof.

2. The method of claim 1, wherein the gene therapy vector comprises a viral vector.

3. The method of claim 2, wherein the viral vector comprises one or more of AAV1, AAV2, AAV3, AAV4, AAV5, AAV6, AAV7, AAV8, AAV9, AAV10, AAV11, AAV12, variations thereof, and combinations thereof.

4. The method of claim 2, wherein the viral vector comprises AAV6 or AAV9.

5. (canceled)

6. The method of claim 1, wherein the nucleic acid sequence further encodes for a cardiac-specific promoter.

7. The method of claim 1, wherein the therapeutic dose is effective to treat or prevent arrhythmogenic right ventricular cardiomyopathy (ARVC) by effecting production of the PKP2 or a functional variant thereof by the cardiomyocytes of the human subject.

8. The method of claim 1, wherein the delivering of the therapeutic dose is performed intravenously.

9. A gene therapy vector adapted for expressing a nucleic acid sequence within cardiomyocytes of a human subject, the nucleic acid sequence comprising:
a first sequence encoding for PKP2 or a functional variant thereof; and
a second sequence comprising a cardiac-specific promoter.

10. (canceled)

11. The gene therapy vector of claim 9 , wherein the gene therapy vector comprises a viral vector, and wherein the viral vector comprises one or more of AAV1, AAV2, AAV3, AAV4, AAV5, AAV6, AAV7, AAV8, AAV9, AAV10, AAV11, AAV12, variations thereof, and combinations thereof.

12. The gene therapy vector of claim 11, wherein the viral vector comprises AAV6 or AAV9.

13. The gene therapy vector of claim 9, wherein the cardiac-specific promoter comprises TNNT2.

14. A therapeutic formulation for treating or preventing cardiomyopathy in a human subject, the therapeutic formulation comprising:
a pharmaceutically acceptable excipient or carrier; and
a viral vector comprising a nucleic acid sequence encoding for PKP2 or a functional variant thereof.

15. The therapeutic formulation of claim 14, further comprising:
one or more additional viral vectors each comprising a nucleic acid sequence encoding for one or more non-PKP2 sarcomeric proteins or functional variants thereof

16. A method of genetically modifying a PKP2-mutated cardiomyocyte to express non-mutated PKP2, the method comprising:
transfecting the PKP2-mutated cardiomyocyte with a nucleic acid sequence that encodes for the non-mutated PKP2.

17. The method of claim 16, wherein the nucleic acid sequence is delivered via a viral vector comprises AAV6 or AAV9.

18. (canceled)

19. The method of claim 16, wherein the nucleic acid sequence further encodes for a cardiac-specific promoter, and wherein the cardiac-specific promoter comprises TNNT2.

20. (canceled)

21. The method of claim 16, wherein the PKP2 is PKP2 isoform 2a.

22. The method of claim 16, wherein the PKP2 is PKP2 isoform 2b.

23. The gene therapy vector or therapeutic formulation of claim 9, wherein the PKP2 is PKP2 isoform 2a.

24. The gene therapy vector or therapeutic formulation of claim 9, wherein the PKP2 is PKP2 isoform 2b.


SEQUENCE LISTING (6 sequences)

SEQ ID NO: 1 - DNA, 2764 bp, Homo sapiens
gagtccagaggcaggcgagcagctcggtcgcccccaccggccccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgaggaaaatgacaaagtattctcggctgcaaaaatccccaaaggaaaacacctatttttctactacccagcccaagaaacctcaaaagcatgccttgtttctatccttctctatttccgtggtcccctgaatccagaaaacaaatagaacataattttatgagtcttccagaagacctttgcaagtttgccaccagtagataccggcc

SEQ ID NO: 2 - protein, 837 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 3 - DNA, 2643 bp, artificial
  PKP2b codon optimized
atggccgcccccggagcacctgccgagtatggctacattcgcaccgtcctgggacagcagattctgggacagctggattcatcaagcctggccctgccttctgaggccaagctgaagctggcaggaagctccggaaggggaggacagaccgtgaagagcctgagaatccaggagcaggtgcagcagacactggcccggaagggcagatctagcgtgggcaacggcaatctgcacaggacctcctctgtgccagagtacgtgtataacctgcacctggtggagaatgacttcgtgggaggccgcagcccagtgccaaagacatacgatatgctgaaggccggcaccacagcaacctatgagggcaggtggggaagaggaacagcacagtacagctcccagaagtctgtggaggagcggagcctgagacaccctctgcggagactggagatcagcccagactctagccctgagagggcacactatacccactccgattaccagtattctcagagaagccaggcaggacacacactgcaccaccaggagagcaggagggccgccctgctggtgccacctagatacgcccgctctgagatcgtgggcgtgagcagggcaggaaccacatcccggcagagacacttcgacacctaccacagacagtatcagcacggctctgtgagcgacacagtgtttgattccatccctgccaacccagccctgctgacctatcctcggccaggcacatccagatctatgggcaacctgctggagaaggagaattacctgaccgcaggcctgacagtgggacaggtgaggcccctggtgcctctgcagccagtgacccagaatcgggccagcagatcctcttggcaccagagctccttccactctaccaggacactgagggaggcaggaccaagcgtggcagtggactctagcggccggagagcccacctgaccgtgggacaggcagcagcaggaggatccggcaacctgctgacagagaggtccacctttacagactctcagctgggcaatgccgatatggagatgaccctggagagggccgtgagcatgctggaggcagaccacatgctgccatccaggatctctgccgcagccacattcatccagcacgagtgctttcagaagtccgaggcaaggaagagggtgaaccagctgaggggcatcctgaagctgctgcagctgctgaaggtgcagaacgaggatgtgcagagggccgtgtgcggcgccctgaggaatctggtgttcgaggacaacgataataagctggaggtggcagagctgaacggagtgccaaggctgctgcaggtgctgaagcagacccgcgacctggagacaaagaagcagatcaccgatcacacagtgaacctgcggagcagaaatggatggcctggagcagtggcacacgcatgcaatccaagcaccctgggaggacagggaggaaggatcacaagatccggcgtgcgggaccagcctgatcagcacggcctgctgtggaacctgtcctctaatgacaagctgaagaacctgatgatcaccgaggccctgctgaccctgacagagaatatcatcatcccttttagcggctggccagagggcgattatcccaaggccaacggcctgctggacttcgatatcttttacaacgtgaccggctgcctgaggaatatgagctccgccggagcagacggaagaaaggccatgaggcgctgtgacggcctgatcgattccctggtgcactacgtgcggggcaccatcgccgattatcagcccgacgataaggccacagagaactgcgtgtgcatcctgcacaatctgtcttatcagctggaggccgagctgcctgagaagtacagccagaacatctatatccagaacagaaatatccagaccgacaacaataagagcatcggctgcttcggcagcaggtcccgcaaggtgaaggagcagtaccaggatgtgcccatgcctgaggagaagtccaatcccaagggcgtggagtggctgtggcactctatcgtgatcaggatgtatctgagcctgatcgccaagtccgtgcgcaactacacccaggaggcatctctgggcgccctgcagaatctgacagcaggatctggaccaatgcccaccagcgtggcccagacagtggtgcagaaggagtccggcctgcagcacacccggaagatgctgcacgtgggcgacccatccgtgaagaagacagccatctctctgctgaggaacctgagccgcaatctgtccctgcagaacgagatcgccaaggagacactgcccgatctggtgagcatcatcccagacaccgtgccctccacagatctgctgatcgagacaacagcctccgcctgttacaccctgaacaatatcatccagaactcttatcagaatgcccgggacctgctgaacacaggcggcatccagaagatcatggcaatctccgccggcgatgcatacgcatctaataaggccagcaaggccgcctccgtgctgctgtattctctgtgggcacacaccgagctgcaccacgcatacaagaaggcccagtttaagaagactgatttcgtgaatagcagaacagccaaagcctaccacagcctgaaggac

SEQ ID NO: 4 - protein, 881 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITDHTVNLRSRNGWPGAVAHACNPSTLGGQGGRITRSGVRDQPDQHGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 5 - DNA, 600 bp, Homo sapiens
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 6 - DNA, 8383 bp, artificial
  Exemplary vector expressing PKP2b
ggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggtaccggtcgccaccatggactacaaagaccatgacggtgattataaagatcatgacatcgattacaaggatgacgatgacaagcttggtaccgagctcggatccatggccgcccccggagcacctgccgagtatggctacattcgcaccgtcctgggacagcagattctgggacagctggattcatcaagcctggccctgccttctgaggccaagctgaagctggcaggaagctccggaaggggaggacagaccgtgaagagcctgagaatccaggagcaggtgcagcagacactggcccggaagggcagatctagcgtgggcaacggcaatctgcacaggacctcctctgtgccagagtacgtgtataacctgcacctggtggagaatgacttcgtgggaggccgcagcccagtgccaaagacatacgatatgctgaaggccggcaccacagcaacctatgagggcaggtggggaagaggaacagcacagtacagctcccagaagtctgtggaggagcggagcctgagacaccctctgcggagactggagatcagcccagactctagccctgagagggcacactatacccactccgattaccagtattctcagagaagccaggcaggacacacactgcaccaccaggagagcaggagggccgccctgctggtgccacctagatacgcccgctctgagatcgtgggcgtgagcagggcaggaaccacatcccggcagagacacttcgacacctaccacagacagtatcagcacggctctgtgagcgacacagtgtttgattccatccctgccaacccagccctgctgacctatcctcggccaggcacatccagatctatgggcaacctgctggagaaggagaattacctgaccgcaggcctgacagtgggacaggtgaggcccctggtgcctctgcagccagtgacccagaatcgggccagcagatcctcttggcaccagagctccttccactctaccaggacactgagggaggcaggaccaagcgtggcagtggactctagcggccggagagcccacctgaccgtgggacaggcagcagcaggaggatccggcaacctgctgacagagaggtccacctttacagactctcagctgggcaatgccgatatggagatgaccctggagagggccgtgagcatgctggaggcagaccacatgctgccatccaggatctctgccgcagccacattcatccagcacgagtgctttcagaagtccgaggcaaggaagagggtgaaccagctgaggggcatcctgaagctgctgcagctgctgaaggtgcagaacgaggatgtgcagagggccgtgtgcggcgccctgaggaatctggtgttcgaggacaacgataataagctggaggtggcagagctgaacggagtgccaaggctgctgcaggtgctgaagcagacccgcgacctggagacaaagaagcagatcaccgatcacacagtgaacctgcggagcagaaatggatggcctggagcagtggcacacgcatgcaatccaagcaccctgggaggacagggaggaaggatcacaagatccggcgtgcgggaccagcctgatcagcacggcctgctgtggaacctgtcctctaatgacaagctgaagaacctgatgatcaccgaggccctgctgaccctgacagagaatatcatcatcccttttagcggctggccagagggcgattatcccaaggccaacggcctgctggacttcgatatcttttacaacgtgaccggctgcctgaggaatatgagctccgccggagcagacggaagaaaggccatgaggcgctgtgacggcctgatcgattccctggtgcactacgtgcggggcaccatcgccgattatcagcccgacgataaggccacagagaactgcgtgtgcatcctgcacaatctgtcttatcagctggaggccgagctgcctgagaagtacagccagaacatctatatccagaacagaaatatccagaccgacaacaataagagcatcggctgcttcggcagcaggtcccgcaaggtgaaggagcagtaccaggatgtgcccatgcctgaggagaagtccaatcccaagggcgtggagtggctgtggcactctatcgtgatcaggatgtatctgagcctgatcgccaagtccgtgcgcaactacacccaggaggcatctctgggcgccctgcagaatctgacagcaggatctggaccaatgcccaccagcgtggcccagacagtggtgcagaaggagtccggcctgcagcacacccggaagatgctgcacgtgggcgacccatccgtgaagaagacagccatctctctgctgaggaacctgagccgcaatctgtccctgcagaacgagatcgccaaggagacactgcccgatctggtgagcatcatcccagacaccgtgccctccacagatctgctgatcgagacaacagcctccgcctgttacaccctgaacaatatcatccagaactcttatcagaatgcccgggacctgctgaacacaggcggcatccagaagatcatggcaatctccgccggcgatgcatacgcatctaataaggccagcaaggccgcctccgtgctgctgtattctctgtgggcacacaccgagctgcaccacgcatacaagaaggcccagtttaagaagactgatttcgtgaatagcagaacagccaaagcctaccacagcctgaaggacctcgagggatctggagcaacaaacttctcactactcaaacaagcaggtgacgtggaggagaatcccgggcctaagcttatgaaaaccttcaacatctctcagcaggatctggagctggtggaggtcgccactgagaagatcaccatgctctatgaggacaacaagcaccatgtcggggcggccatcaggaccaagactggggagatcatctctgctgtccacattgaagcctacattggcagggtcactgtctgtgctgaagccattgccattgggtctgctgtgagcaacgggcagaaggactttgacaccattgtggctgtcaggcacccctactctgatgaggtggacagatccatcagggtggtcagcccctgtggcatgtgtagagagctgatctctgactatgctcctgactgctttgtgctcattgagatgaatggcaagctggtcaaaaccaccattgaggaactcatccccctcaagtacaccaggaactaataagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagatgtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagagctcggatcctgagaacttcagggtgagtctatgggac